(19) United States
(12) Patent Application Publication
(10) Pub. No.: US 2010/0016710 A1
(43) Pub. Date: Jan. 21, 2010

(54) PROSTATE TREATMENT APPARATUS

(76) Inventors: Dinesh Kumar, Grass Valley, CA (US); Jasjit S. Suri, Roseville, CA (US)

Correspondence Address: MARSH, FISCHMANN & BREYFOGLE LLP, 8055 East Tufts Avenue, Suite 450, Denver, CO, 80237, US

(21) Appl. No.: 12/501,943

(22) Filed: Jul. 13, 2009

Related U.S. Application Data
(60) Provisional application No. 61/080,124, filed on Jul. 11, 2008.

(51) Int. Cl. A61B 5/05

(52) U.S. Cl. 600/425

(57) ABSTRACT

The presented invention aims to address these and/or additional issues by providing a tracked grid for brachytherapy applications that can adjust to changes in the prostate between original image acquisition. The grid is allowed to be moved around and even rotated under tracked conditions, such that it can be maneuvered to align with any target region. A virtual 3-D grid is displayed on a computer with respect to the actual anatomy of the patient. The tracked grid can be moved relative to the virtual 3-D grid, such that the brachytherapy, cryo therapy or any other type of image guided therapy may be performed in real 3-D or even 4-D, when motion compensation is embedded during the procedure.

### CROSS REFERENCE TO RELATED APPLICATIONS

This application claims the benefit of U.S. Provisional No. 61/080,124, entitled: “PROSTATE TREATMENT APPARATUS,” having a filing date of Jul. 11, 2008, the entire contents of which are incorporated by reference herein.

### FIELD

The present application relates to image guided surgery. One aspect of the application is directed to image guided therapy for treatment of prostate cancer.

### BACKGROUND

Prostate cancer is the most common type of cancer found in American men, other than skin cancer. According to American Cancer Society, there will be about 234,460 new cases of prostate cancer in the United States in 2006 and about 27,350 men will die of this disease. Prostate cancer is only curable at an early stage. Therefore, early detection is extremely important to reduce mortality and enhance the cure rate.

Once prostate cancer is detected, treatment options historically favored removal of the prostate. However, recent developments in targeted focal therapies has allowed for targeting cancerous cells without prostate removal. For instance, brachytherapy is an established method for treatment of prostate cancer. In this method, a needle carrying a number of radioactive beads (or seeds) are guided and placed in suspected/established cancer locations, typically using a two-dimensional (2-D) trans-rectal ultrasound (TRUS) guidance. The beads cause death of surrounding cells in a small neighborhood and a number of beads are required as per the dosimetry requirements. The beads may be placed along a straight line as the needle is retracted. The TRUS probe is generally rotated to keep the needle in field of view as it is entered trans-perineally via an external brachy “grid” near the perineum.

Such an existing brachy grid device is illustrated in FIG. 1. As shown, the device provides a physical grid with holes provided for a needle containing seeds to be inserted trans-perineally. The physical grid is kept fixed by support equipment such that the grid does not move at all during the procedure. However, as per dosimetry plans, the line of beads may be different from actual 2-D locations or line of sight that the grid provides. This may be due to any of the reasons above as well as due to distribution of cancer cells or tissue necrosis. This limitation may result in overtreatment, where the user chooses to add more seeds because frame of reference has changed, or under treatment where the seeds are placed at wrong locations, resulting in death of healthy tissues while not affecting the target cancer cells.

Further, the beads move as the needle is retracted owing to needle pressure/friction against the tissue. Another cause of the seed movement is the patient movement during the procedure. More important is that during the therapy procedure, pubic bone can interfere. As a result, the physician has to insert fingers in the rectum canal during the procedure. This can further cause movement of the prostate and the seeds. In the prior art method of seed insertion, the ultrasound positioning device (so called ultrasound probe) and the needle are inserted from different anatomic locations such as rectum canal and perineum. The imaging probe images the prostate in transverse and longitudinal planes while the needle is inserted along the longitudinal (sagittal) plane. This causes difficulty in understanding the in-plane movement of the needle tip. The 3^{rd} dimension is thus totally obscured during the therapy treatment and hence placement of the seeds for treatment is very difficult. Missing 3D spatial information brings a major challenge besides the motion of the prostate. Combined effect of missing spatial and temporal information during real-time treatment brings major challenge during treatment process. Further, the beads migrate into other tissues or may even enter the urethra, where the patient may pass it through urine.

Finally, it is noted that brachy locations may be planned using original images acquired hours or weeks before a treatment procedure. That is, there may be a time gap of up to weeks between a 3D scanning procedure and an actual treatment procedure. During this time, frame of reference for the prostate changes. In addition, there could be other changes due to patient positioning, abdominal contents and muscular tension. Patient breathing motion also may complicate the workflow while performing the procedure.

### SUMMARY

The presented invention aims to address these and/or additional issues by providing a tracked grid for brachytherapy applications that can adjust to changes in the prostate between original image acquisition. The grid is allowed to be moved around and even rotated under tracked conditions, such that it can be maneuvered to align with any target region. A virtual 3-D grid is displayed on a computer with respect to the actual anatomy of the patient. The tracked grid can be moved relative to the virtual 3-D grid, such that the brachytherapy, cryo therapy or any other type of image guided therapy may be performed in real 3-D or even 4-D, when motion compensation is embedded during the procedure. Compared with 2-D techniques which exists now, where the external grid provides a system for placing seeds along parallel and restricted paths the presented system permits improved freedom and improved focal targeting. While the presented utilities primarily discuss brachytherapy in details, the same methods can be applied directly with very little modifications to other prostate cancer treatment methods such as cryo therapy or ablation using thermal applicators.

In one aspect, an improved system and method (i.e., utility) for image guided brachytherapy procedure is presented. The presented system uses a treatment plan planned using a 3-D imaging modality such as TRUS, MRI or CT and customizes it to the patient's anatomy during the procedure by taking into account the change in frame of reference owing to non-rigid deformation of soft tissues between the earlier TRUS, CT or MRI scan and the procedure and during the procedure. The system includes a 3-D TRUS scanning subsystem for 3-D ultrasound scan just prior the procedure. The system includes a graphical user interface for displaying a 3-D images of the patient's anatomy and also provides a virtual grid relative to the anatomy. Preferably, this display also displays a live ultrasound 3-D motion compensated image and/or one or more treatment plans with respect to the virtual grid. Finally, the system includes a tracked brachytherapy grid for aligning the needle trajectory with a planned location. The tracked grid is movable relative to the virtual grid such that a needle aperture in the tracked grid may be aligned with target locations within the virtual gird.

In one arrangement, the utility includes motion compensation engine for removing motion artifacts during the procedure. In another arrangement, an image fusion engine is provided for customizing a plan based on MRI/CT images to the current patient anatomy.

In another aspect, an improved utility is provided for performing image guided brachytherapy. The presented system uses a treatment plan planned using a 3-D imaging modality such as TRUS, MRI or CT and customizes it to the patient's anatomy during the procedure by taking into account the change in frame of reference owing to non-rigid deformation of soft tissues between the earlier TRUS, CT or MRI scan and the procedure and during the procedure. 3-D TRUS images are acquired just before the procedure to identify the current frame of reference of patient anatomy just before the procedure. The plans are defined with respect to 3-D images acquired previously using TRUS, CT or MRI imaging technique are deformed to the current geometry and shape the patient anatomy.

In another aspect, an improved utility is provided for performing image guided brachytherapy. The presented system uses a treatment plan planned using a 3-D imaging modality such as TRUS, MRI or CT and customizes it to the patient's anatomy during the procedure by taking into account the change in frame of reference owing to non-rigid deformation of soft tissues between the earlier TRUS, CT or MRI scan and the procedure and during the procedure. The system also contains a subsystem for image fusion. The plans are defined in coordinate system of MRI or CT images acquired hours to weeks before the actual procedure. The 3-D TRUS, MRI or CT images used for computing the plan are deformed into the current shape of prostate by registering with 3-D TRUS image acquired by the presented system. The registration parameters are used to transform the plan into the actual frame of reference of patient during the procedure, thus resulting in more accurate dose planning.

In another aspect, an improved utility is provided for performing image guided brachytherapy. The presented system uses a treatment plan planned using a 3-D imaging modality such as TRUS, MRI or CT and customizes it to the patient's anatomy during the procedure by taking into account the change in frame of reference owing to non-rigid deformation of soft tissues between the earlier TRUS, CT or MRI scan and the procedure and during the procedure. The system also contains a subsystem for image fusion. The plans are defined in coordinate system of TRUS, MRI or CT images acquired hours to weeks before the actual procedure. The 3-D TRUS, MRI or CT images used for computing the plan are deformed into the current shape of prostate by registering with 3-D TRUS image acquired by the presented system. The registration parameters are used to transform the plan into the actual frame of reference of patient during the procedure, thus resulting in more accurate dose planning. In addition, the deformed TRUS, MRI and CT images can be displayed such that there is additional information available to the user without the change in coordinate system.

In another aspect, an improved utility is provided for performing image guided brachytherapy. The presented system uses a treatment plan planned using a 3-D imaging modality such as TRUS, MRI or CT and customizes it to the patient's anatomy during the procedure by taking into account the change in frame of reference owing to non-rigid deformation of soft tissues between the earlier TRUS, CT or MRI scan and the procedure and during the procedure. The system also contains a tracked biopsy grid. The grid may just be a needle holder that can be manipulated to align the needle trajectory with the targeted location. This allows the user more flexibility to target regions not lying in line with the existing rigidly mounted brachytherapy grids. This is a significant improvement over the current systems, which ignore the physical changes in prostate and anatomy between the plan and the procedure. In addition, the disadvantages associated with a rigid grid of limited number of points for needle to go through are taken care of, thus providing an accurate and easy to use system.

In another aspect, an improved utility is provided for performing image guided brachytherapy. The presented system uses a treatment plan planned using a 3-D imaging modality such as TRUS, MRI or CT and customizes it to the patient's anatomy during the procedure by taking into account the change in frame of reference owing to non-rigid deformation of soft tissues between the earlier TRUS, CT or MRI scan and the procedure and during the procedure. The system contains a motion compensation subsystem for removing the motion artifacts from the plan during the procedure. This system ensures that the planned sites move along with the patient's anatomy such that they always correspond to the actual target locations with respect to the anatomy and not with respect to a virtual frame of reference of an image acquired earlier.

In another aspect, an improved utility is provided for performing image guided brachytherapy. The presented system uses a treatment plan planned using a 3-D imaging modality such as TRUS, MRI or CT and customizes it to the patient's anatomy during the procedure by taking into account the change in frame of reference owing to non-rigid deformation of soft tissues. In addition to the features listed earlier, the system provides a “virtual” 3-D grid such that the prostate scan can be fit inside this grid along with the plan. The grid represents a fixed 3-D coordinate system, while the image inside it represents a moving frame of reference, as it is tied to the anatomy of the patient. The plans and the image are continuously deformed as a result of motion compensation with respect to the fixed grid, thus providing an accurate representation of actual anatomy so as to provide better dose delivery.

In another aspect, an improved utility is provided for performing image guided brachytherapy. The presented system uses a treatment plan planned using a 3-D imaging modality such as TRUS, MRI or CT and customizes it to the patient's anatomy during the procedure by taking into account the change in frame of reference owing to non-rigid deformation of soft tissues. The system may not only be used for placing permanent seeds, but also may be used for serial or one time brachytherapy treatment using applicator. The tracked brachytherapy grid in this case is replaced by tracked applicator.

In another aspect, an improved utility is provided for performing image guided brachytherapy. The presented method provides capability to enhance plan by using a probabilistic cancer atlas for guidance in planning. The atlas contains real statistics from clinical data for clinically significant cancer cases and shows probabilities of their distribution within prostate. This information can be used to plan such that high probability regions are included in plan even if they were not sampled during a biopsy and pathological records do not exist for those locations. This reduces likelihood of under treatment while also keeping the treatment focused.

### BRIEF DESCRIPTION OF THE FIGURES

FIG. 1 illustrates a prior art brachytherapy grid.

FIG. 2 illustrates a 3D transrectal ultrasound acquisition system.

FIG. 3 illustrates a 3-D model of a prostate.

FIG. 4 illustrates a tracked grid relative to a 2-D image.

FIG. 5 illustrates a virtual grid overlain on a prostate image.

FIG. 6 illustrates a virtual grid overlaid on a prostate image with target locations superimposed.

FIG. 7 illustrates using the virtual grid and tracked grid to guide to a target location.

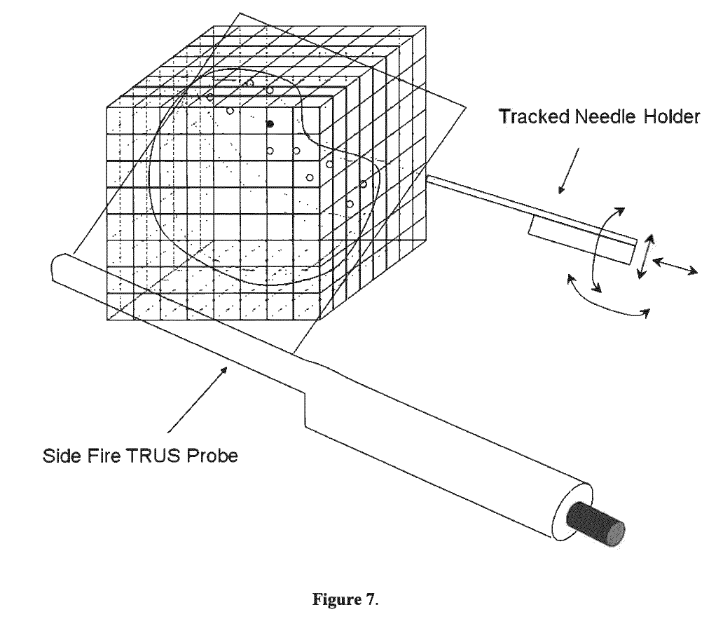

FIG. 8 illustrates a process flow sheet.

FIG. 9 illustrates a process flow sheet.

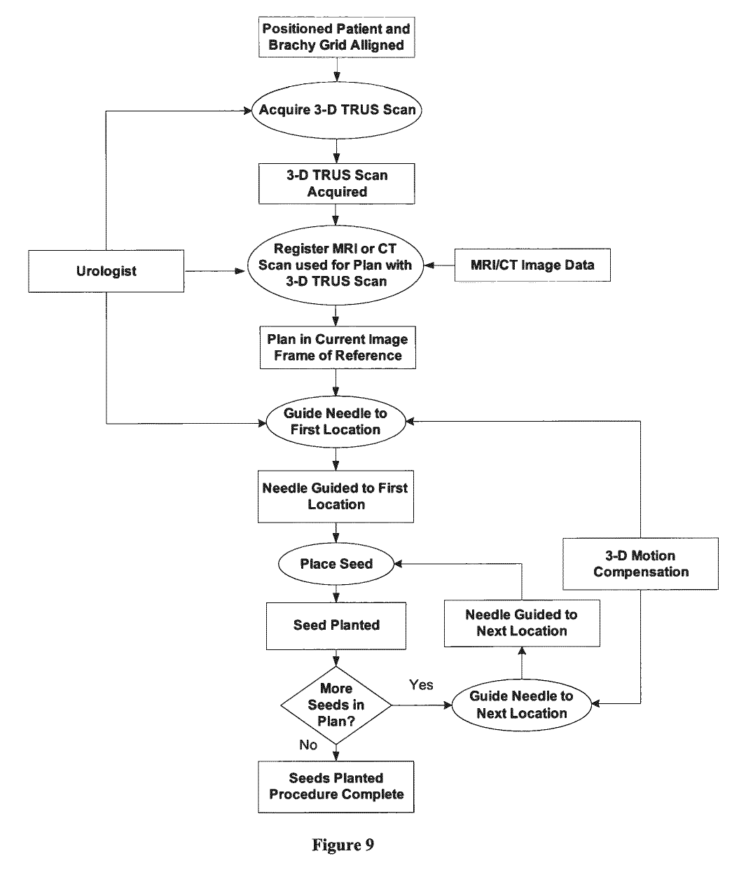

FIG. 10 illustrates a process flow sheet.

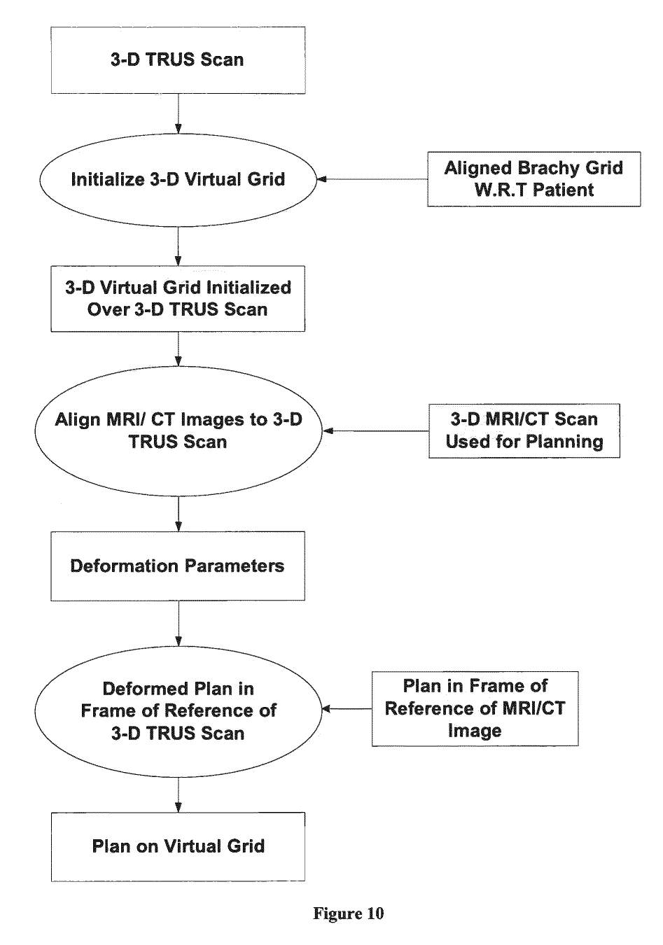

FIG. 11 illustrates a process flow sheet.

FIG. 12 illustrates a process flow sheet.

### DETAILED DESCRIPTION

Reference will now be made to the accompanying drawings, which assist in illustrating the various pertinent features of the various novel aspects of the present disclosure. Although the invention is described primarily with respect to an ultrasound imaging embodiment, the invention is applicable to other imaging modalities, including MRI, CT, and PET, which are applicable to organs and/or internal body parts of humans and animals. In this regard, the following description is presented for purposes of illustration and description. Furthermore, the description is not intended to limit the invention to the form disclosed herein. Consequently, variations and modifications commensurate with the following teachings, and skill and knowledge of the relevant art, are within the scope of the present invention.

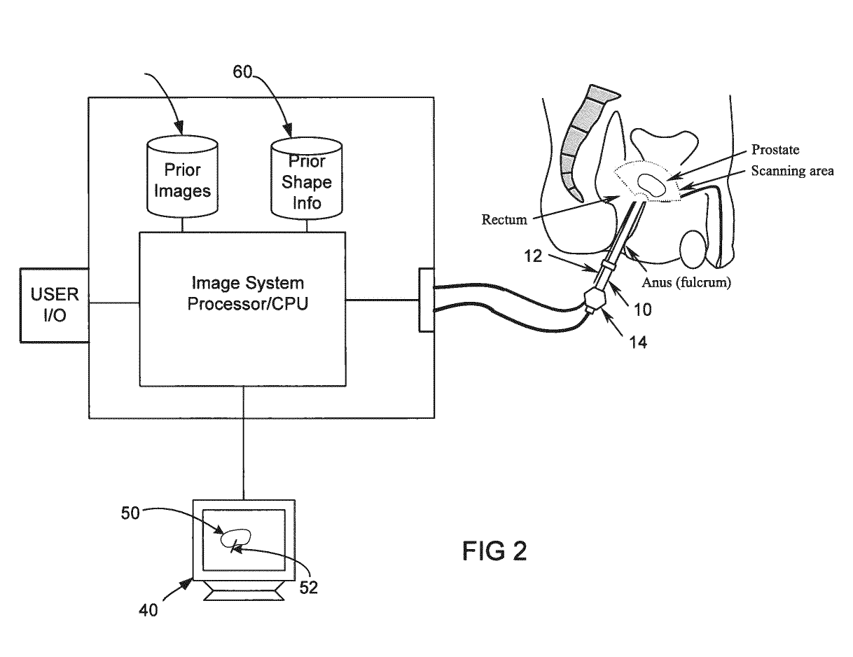

FIG. 2 illustrates an image acquisition system 30. Initially, an exemplary embodiment of the invention will be described in relation to performing prostate biopsy using transrectal ultrasound (TRUS) guidance. As shown in FIG. 2, an ultrasound probe 10 has a biopsy needle assembly 12 attached to its shaft inserted into the rectum from the patient's anus. The biopsy needle is optional and in situations where brachytherapy is performed using the tracked grid discussed herein, the needle need not be included. The illustrated probe 10 is an end-fire transducer that has a scanning area of a fan shape emanating from the front end of the probe (shown as a dotted outline). The probe handle is held by a robotic arm (not shown) that has a set of position sensors 14. These position sensors 14 are connected to the computer 20 of the imaging system 30 via an analog to digital converter. Hence, the computer 20 has real-time information of the location and orientation of the probe 10 in reference to a unified Cartesian (x, y, z) coordinate system.

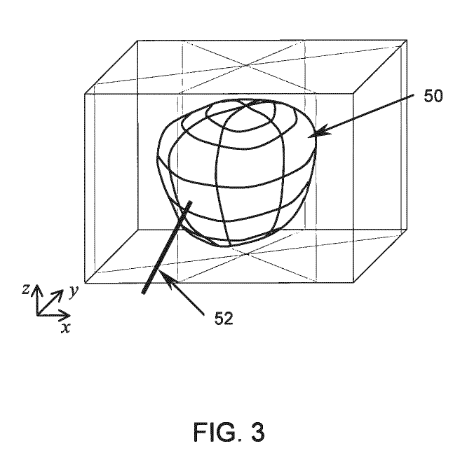

With the dimensions of the probe 10 taken into the calculations, the 3D position of the 2D image planes and/or the needle tip and its orientation is known. The ultrasound probe 10 sends signal to the image guidance system 30, which may be connected to the same computer (e.g., via a video image grabber) as the output of the position sensors 14. In the present embodiment, this computer is integrated into the imaging system 30. The computer 20 therefore has real-time 2D images of the scanning area in memory 22. The image coordinate system and the robotic arm coordinate system are unified by a transformation. Using the acquired 2D images, a prostate surface 50 (e.g., 3D model of the organ) is generated and displayed on a display screen 40 in real-time. See FIG. 3. A biopsy needle may also be modeled on the display, which has a coordinate system so the doctor has the knowledge of the exact locations of the needle and the prostate.

The computer system runs application software and computer programs which can be used to control the system components, provide user interface, and provide the features of the imaging system. The software may be originally provided on computer-readable media, such as compact disks (CDs), magnetic tape, or other mass storage medium. Alternatively, the software may be downloaded from electronic links such as a host or vendor website. The software is installed onto the computer system hard drive and/or electronic memory, and is accessed and controlled by the computer's operating system. Software updates are also electronically available on mass storage media or downloadable from the host or vendor website. The software, as provided on the computer-readable media or downloaded from electronic links, represents a computer program product usable with a programmable computer processor having computer-readable program code embodied therein. The software contains one or more programming modules, subroutines, computer links, and compilations of executable code, which perform the functions of the imaging system. The user interacts with the software via keyboard, mouse, voice recognition, and other user-interface devices (e.g., user I/O devices) connected to the computer system.

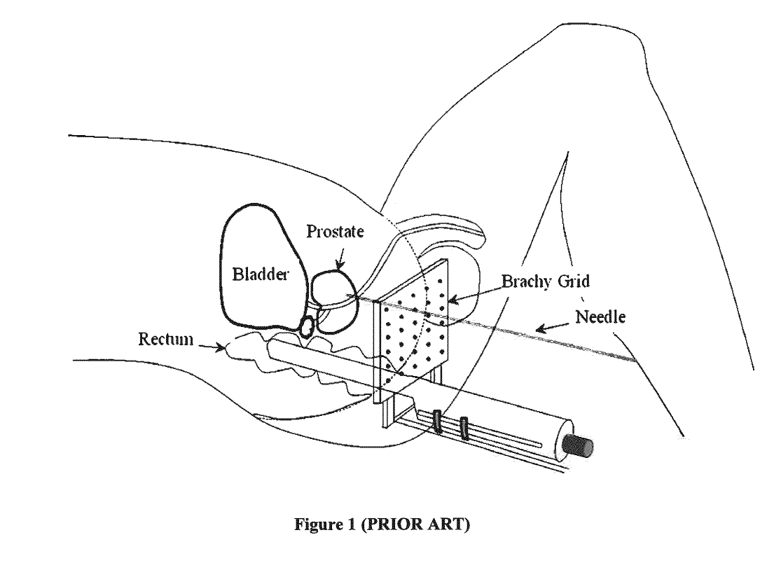

As noted above, in one prior art prior method, a prior ultrasound image is used to plan brachytherapy seed locations and guide a needle to that position. FIG. 1 shows a typical setup, where, with patient in lithotomic position, a side fire TRUS probe mounted on a stepper is inserted into rectum of the patient while a tilting grid is fixed relative to the probe. The needle containing the seeds is inserted using 3D TRUS guidance and the needle is segmented to compute the insertion depth and deflection using live image recorded while insertion. If the needle appears to bend more than acceptable level, it is withdrawn and reinserted. Likewise, after the placement of seeds, the plan is updated using dosimetry computations again. While planning using an imaging modality like MRI and CT has been in place for the purpose of planning, the main advantage of the prior art is in usage of 3-D ultrasound as ultrasound machines are easily movable and inexpensive. In addition, the user may rescan 3-D image anytime during the scan. However, the method still suffers from limitations. For instance, the method uses traditional grid for aligning needle with the target location. This limits the freedom of placement of the seeds at the planned site. Thus, the plan will need to be recalculated and reevaluated every time if the patient moves.

As discussed herein, the current application includes a movable tracked grid with one hole for needle guidance where the grid can be manipulated relative to the patient within a large range of motion. This tracked grid moves relative to a virtual grid that is superimposed on the 3D image, which allows, inter alia, compensation patient movement. The movable tracked grid allows a brachytherapy needle (or other targeted therapy device) to be aligned more accurately due to more degrees of freedom, which permits to needle to be positioned at a desired location very precisely. More than that, the user does not have to restrict his insertion in a fixed systematic fashion using conventional grid method. Control of the needle guidance system allows high precision in an uncertain system having motion.

Another limitation of the prior art device is that the computation of needle trajectory is highly dependent upon needle segmentation, which is difficult and indirect solution. The current application improves this by directly measuring the needle trajectory instead of computing it from segmentation of noisy ultrasound images. This is done via tracked grid, which provides the orientation of hole through which needle is passed. The orientation and position of the tracking grid are always known through tracking. Since there is only one hole in the grid which needle has to pass through, the trajectory of needle always aligns with the orientation of the hole in the tracking grid. Using this information along with needle segmentation provides more robust and accurate method for target placement and computation of actual locations of beads placed. As the tracking information of grid is processed, the projected needle trajectory is displayed to the user such that while aligning the grid, the user is able to see where the needle will pass through. This also means that the user may place the needle in any arbitrary orientation and still be able to see the projected trajectory in 3-D space and in frame of reference of a virtual grid.

The inventors of previous art are mainly concerned about “conformal” treatment, which means total ablation of the prostate. The presented invention aims at addressing this by allowing user to perform targeted therapy. Like the pervious technology, the presented invention can be applied to other forms of treatment including but not limited to cryotherapy, thermal ablation. The targeted focal therapy is possible due to better tracking and control of needle during the procedure. The previous art does not have ability to display a virtual 3-D grid overlaid on 3-D view. Such a grid is extremely helpful for planning, since the user can place a seed in each grid element of reasonable size to ablate the tissue locally. Thus the current application has more local control, which is the key in precise therapy unlike other therapies where the whole prostate is damaged during the therapy.

The only way of handling motion compensation in the previous art is to rescan the 3-D ultrasound volume during the procedure every time the physician suspects a motion and re-plan the procedure. The current application performs motion compensation and updates the plan accordingly in real-time while also keeping track of motion for already placed seeds, thus easing workflow, while correcting the location for already placed seeds for motion. This is used to update the plan in real time such that the plan always conforms to the current frame of reference and no two frames of reference exist at any one time.

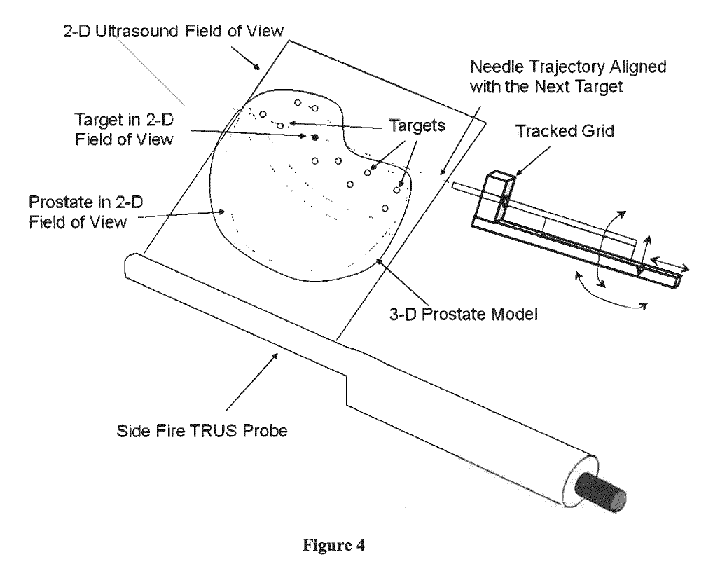

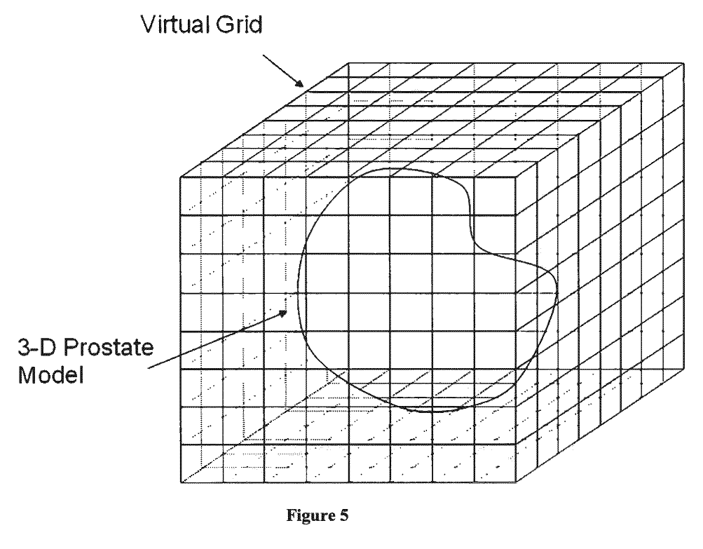

FIGS. 4 and 5 illustrate the procedure using improved method presented in this work. In this method, the brachy grid is much smaller and has only one hole for needle to go through and is mounted on a tracking mechanism (not shown). Such a tracking mechanism is disclosed in co-pending U.S. application Ser. No. 11/650,482 entitled “Tracker Holder Assembly” having a filing date of Sep. 5, 2007, the entire contents of which are incorporated herein by reference. The grid, as such, acts as a needle holder as well. As the grid is manipulated, the orientation of the needle trajectory is computed by the software in near real-time and the trajectory is displayed to the user, relative to the planned location and a virtual grid. The patient is first positioned into examination table and a grid is aligned as per the plan. Once the grid has been aligned to face the prostate for a transperineal brachytherapy procedure, the user refers to the seed plan from the 3-D image (MRI/CT) acquired earlier and places the seeds at the locations based on the plan while using real-time TRUS image for guidance. That is, a mounted TRUS probe is aligned with the field of view of the target according to the target location in the virtual grid. The tracked needle holder is then aligned with the target such that the extended trajectory of the needle passes through the next target. The needle containing the radioactive beads is then inserted so as to place the beads at the desired targets.

In order to perform a procedure using a 3-D image guidance system, a 3-D frame of reference must be established that defines the correspondences between the real world space and the image space, such that information from different sources can be combined together in one common frame of reference. This coordinate system is defined as a virtual grid. The grid is a 3-D space, where each grid element represents a small cubic area. The size and resolution of this grid may be adjustable depending upon the size of organ and resolution required. The virtual grid is defined relative to the frame of reference of acquisition of 3-D volume and does not change after that. The 3-D prostate model is displayed on this grid and may deform between planning and the procedure. In such cases, the model will be deformed such that the plan is always defined correctly in frame of reference of the virtual grid and plan is updated as per the shape changes.

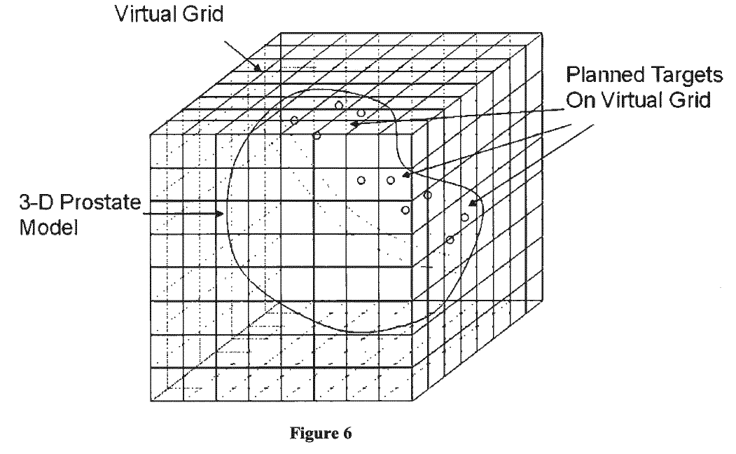

FIG. 5 shows an example of prostate model in a 3-D virtual grid. The grid displays the lines and the prostate is overlaid onto this grid. The prostate model may be achieved by using a segmentation method that extracts the boundaries of prostate in 3-D image. This surface is represented in frame of reference of the virtual grid. Any plan obtained from another modality or planned spontaneously or just before the procedure may then be converted into this frame of reference and shown as an overlay. That is, the virtual grid is overlaid on the 3-D prostate model along with the planned targets. See FIG. 6. The plan is defined relative to the virtual grid such that any changes in anatomy between plan and the procedure are addressed by deforming the prostate model into frame of reference of the virtual 3-D grid. One method for providing such deformation is disclosed in U.S. application Ser. No. 11/750,854 entitled “Repeat Biopsy System” having a filing date of May 18, 2007, the entire contents of which are incorporated herein by reference. As shown, the frame of reference of TRUS probe is fixed with respect to the virtual grid. During the brachytherapy procedure, the 3-D prostate model and the plan locations are continuously updated relative to the virtual grid. A tracked needle is aligned with the next target location and inserted into the gland to place the radioactive beads at that location.

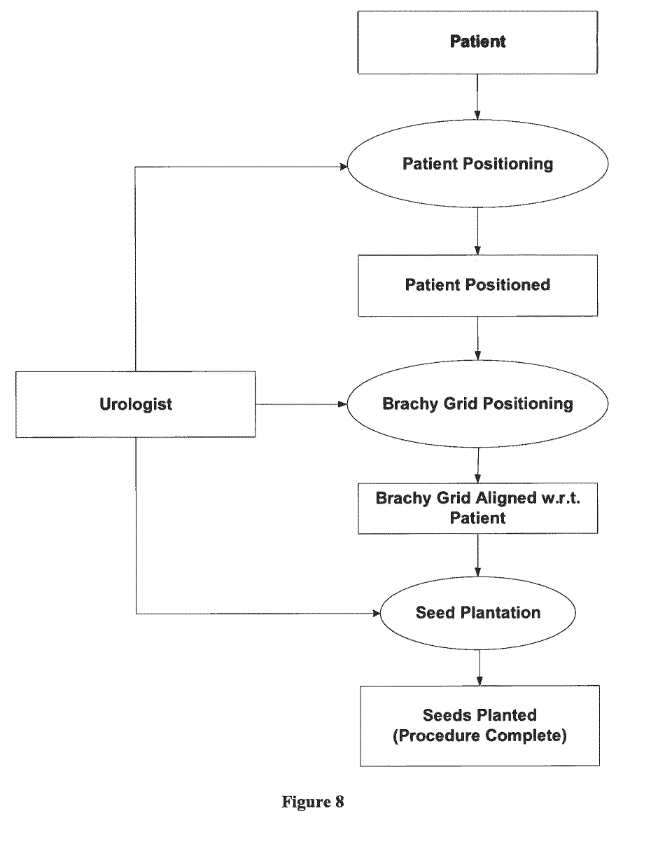

The virtual grid also always shows the tracked needle trajectory based on the tracking information obtained from the tracked grid, such that the user is able to always see where the needle will go if inserted. In addition, the user is also able to see the plane of insertion of needle by manipulating the TRUS probe. This manipulation may be automated or manual. Once the needle trajectory is in plane of view of live image or intersects the live image, the user inserts the needle up to the planned location. The needle holder may contain an attachment for mounting needle such that even the insertion depth of the needle may be computed. Alternatively, the needle is inserted using just live 2-D ultrasound view and the depth is computed using either automated segmented method or tip identified by the user. The information is stored and displayed in frame of reference of the virtual grid as per workflow of FIG. 8.

In the presented method, the grid is reduced to having just one hole, but assigned full degrees of freedom for it to align with a target plan. The grid is mounted on a tracking device, which measures the orientation and position of the physical grid with respect to the virtual grid. Since the grid has just one hole, it may also work as a needle holder with a sliding mechanism that holds the needle as it is inserted and retracted. In such a method, the seed placement can directly be measured by measuring the orientation of needle trajectory and depth of insertion. In such cases, needle segmentation is not required to know the seed position other than due to bending of needle.

FIG. 8 shows the overall procedure for a prostate cancer therapy procedure. The figure shows the workflow for a brachytherapy procedure, but the same method may be applied to any focal treatment method for prostate cancer, such as ablation using thermal applicator or cryoablation. As per the workflow, the urologist positions the patient and acquires the 3-D trans-rectal ultrasound scan. A 3-D virtual grid is defined as the coordinate system in which the 3-D image is placed and the planned locations are imported onto this grid through image fusion. The physical brachy grid (or holder for an applicator) is aligned with respect to the patient so that the needle trajectory aligns with the target location of the plan. The needle is then inserted along the path and when in place, the seeds are planted. All the planned sites are visited as such and seeds are planted at all the planned locations.

Many times, the plan is done based on a good structural imaging modality such as MRI or CT and the actual procedure is performed under 2-D ultrasound guidance. In such cases, the plan from MRI or CT or any other imaging modality can be converted into the 3-D TRUS image used at the time of procedure such that the plan can be easily transformed and defined into coordinates of the virtual grid. The system contains a mutual information and shape based fusion method that fuses the modalities together using an elastic warping method such that the plan is defined in current frame of reference. In addition, the system provides superposition of probabilistic atlas showing likelihoods of cancer based on real clinical statistics. Such an atlas can be extremely useful in deciding a plan once it is established that the patient has cancer. The atlas shows a typical distribution of clinical significant cancers and suggests regions that may need to be ablated. Such a cancer atlas is set forth in U.S. application Ser. No. 11/740,807 entitled “Improved method and system for 3-D biopsy” having a filing date of Apr. 26, 2007, the entire contents of which are incorporated herein by reference.

As mentioned earlier, due to needle insertion or normal movement of anatomical structures, the planned locations may not correspond exactly to the current physical state of the anatomical structure in question (prostate here). The prostate being a soft tissue, surrounded by soft tissues, is a deformable object that tends to move along with its neighboring structures by itself. Likewise, as the needle is inserted, it tends to move along the direction of needle in direction of force, for example, it moves away from the user when the needle is inserted and the needle pulls it along while being retracted. This causes complications as the plan is no longer valid without correction. Hence, the plan must be updated continuously and corrected for these motion artifacts. The presented work contains a system for near real-time motion compensation such that as the prostate moves, the motion parameters are computed and applied to the prostate model and plan in the virtual grid, such that the prostate model and plans are dynamically updated in frame of reference of the virtual grid. This can be done under two different scenarios:

First, the TRUS probe is a 2-dimensional imaging probe. In this case, the 3-D image may be acquired using a freehand tracking mechanism attached to the probe and manipulating the probe such that it scans the entire prostate with reasonable resolution. In such cases, the manipulation may even be motorized to scan the prostate more uniformly. The motion compensation in such cases can be achieved by finding the 2-D slice in current field of view and matching it with a 2-D section from the acquired 3-D scan. The transformation matrix provides the correspondence between the live 2-D image and the extracted 2-D section from the 3-D acquired scan. This method may be made even more robust by keeping a few previous frames in memory buffer of the computer and be used to find the corresponding slices in 3-D. Such an approach reduces the likelihood of getting stuck in a local minimum that is far from the actual location. Second, the TRUS system is 3-D system. In such cases, every few seconds, the prostate will be scanned in a low resolution and a transformation matrix found between the acquired 3-D scan used for plan and the current 3-D scan. This transformation may then be applied to the image coordinate system to synchronize it with the actual physical geometry of the object.

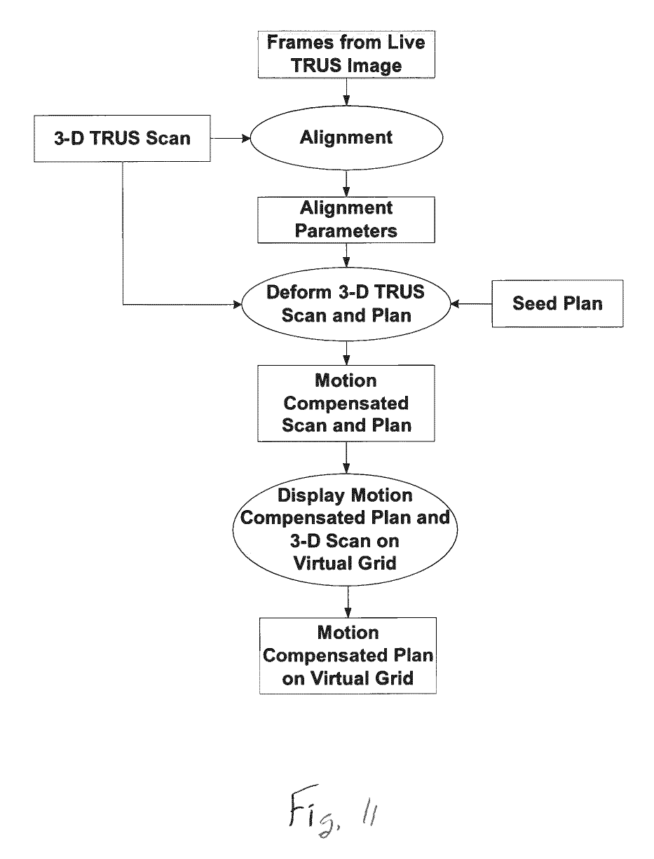

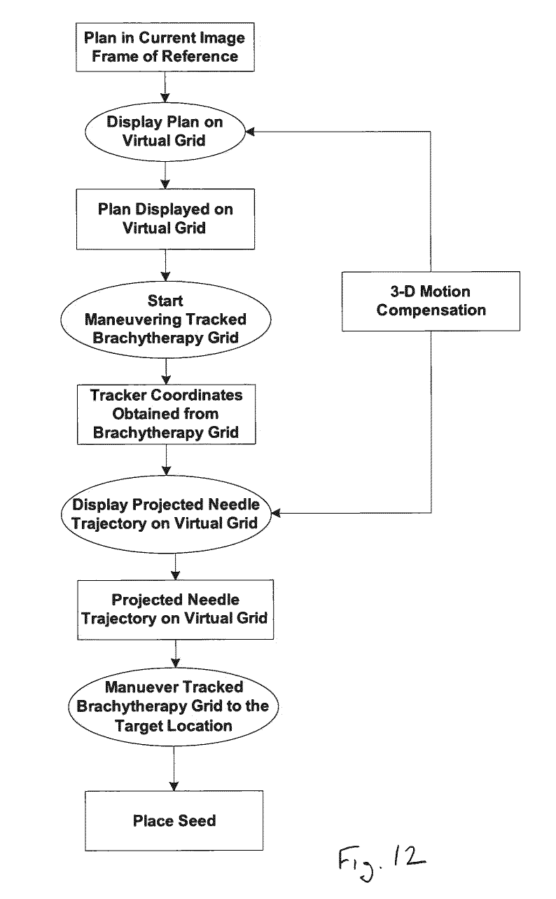

In any case, the system will perform workflow as per motion compensation system shown in FIG. 11. The live images from ultrasound system are aligned with the 3-D image at the beginning of the procedure. The alignment parameters are then applied to the transformation matrix between physical space (live image) and the image space (3-D virtual grid space). Upon updating the transformation, the plan is recomputed and the prostate model is placed according to the computed motion compensating transformation. The current locations of previously places bead points are recomputed and displayed to the user so that the motion artifacts are reduced. FIG. 12 shows the motion compensation procedure in detail.

The illustrated utility provides numerous advantages. While the method has been explained for brachytherapy procedure mainly, it may be applied to many other focal prostate cancer treatment procedures. The method provides fusion capabilities from the same modality (e.g., TRUS) or from another modality such as MRI or CT. As a result, the plan may be made using any structural or functional imaging modality and can be easily imported to align with the live 2-D or 3-D ultrasound image or image volume. The presented utility also solves the problems associated with motion and tissue deformation through a motion compensation mechanism which transforms the plan and rendered 3-D prostate model into space of a virtual grid, which aligns with the physical grid. Further, the previous art does not handle movement of seeds after they have been placed. The presented utility takes this into account by re-computing the plan during the procedure and updating it, if necessary. Use of the moveable grid also provides more degrees of freedom than a fixed the template grid, which allows for a more flexible treatment plan. Perhaps most important, the utility allows for controlled therapy/focal procedure so as to minimize impact of the procedure on patient's lifestyle.

1. An improved system for image guided brachytherapy procedure, comprising:
a 3-D TRUS scanning subsystem for 3-D generating a current 3-D image of a patient anatomy;
an image combination image for overlaying a previous image of the patient anatomy having a therapy plan therein onto the current 3-D image of patient anatomy;
a virtual grid engine for generating a virtual grid on the current image;
graphical user interface showing virtual grid, live ultrasound video, 3-D motion compensated image and the overlaid plan with respect to the virtual grid a tracked brachytherapy grid for aligning a needle trajectory with said virtual grid.

2. The system of claim 1 further comprising:
motion compensation engine for removing motion artifacts during the procedure.

3. The system of claim 1, further comprising:
a cancer atlas for superimposing cancer information onto the current image.
